(19) United States
(12) Patent Application Publication
(10) Pub. No.: US 2016/0310081 A1
(43) Pub. Date: Oct. 27, 2016

(54) MOUSE CONNECTED TO PHYSIOLOGICAL INDEX DETECTION SYSTEM AND DETECTING METHOD

(71) Applicants: HONG FU JIN PRECISION INDUSTRY (WuHan) CO., LTD., Wuhan (CN); HON HAI PRECISION INDUSTRY CO., LTD., New Taipei (TW)

(72) Inventor: DONG-BO WANG, Wuhan (CN)

(21) Appl. No.: 14/716,343

(22) Filed: May 19, 2015

(30) Foreign Application Priority Data
Apr. 23, 2015 (CN) 201510196261.X

(51) Int. Cl. A61B 5/00; A61B 5/01

(52) U.S. Cl. CPC A61B 5/6897; A61B 5/742; A61B 5/7282; A61B 5/01; A61B 5/0008; A61B 5/486; A61B 2560/0462; A61B 2560/0475

(57) ABSTRACT

A physiological index detection system includes a computer and a mouse, the mouse includes a shell and a physiological index sensing unit and communicates with the computer. The computer includes a display screen and a display control unit. The physiological index sensing unit senses physiological characteristics of a living being who touches the mouse and generates an analog physiological index signal. The physiological index detection system processes the analog physiological index signal to obtain a digital equivalent result in health terms. The display control unit displays the result on the display screen and advice concerning an adverse finding. The disclosure also provides a mouse and a physiological index detecting method.

### CROSS-REFERENCE TO RELATED APPLICATIONS

This application claims priority to Chinese Patent Application No. 201510196261.X, filed on Apr. 23, 2015, the contents of which are incorporated by reference herein.

### FIELD

The subject matter herein generally relates to machine-human interfaces.

### BACKGROUND

Computers has been employed in various fields. People can operate the computer with a mouse. The sole function of the mouse is to operate the computer.

### BRIEF DESCRIPTION OF THE DRAWINGS

Implementations of the present technology will now be described, by way of example only, with reference to the attached figures.

FIG. 1 is an isometric view of a physiological index detection system.

FIG. 2 is a block diagram of the physiological index detection system in FIG. 1.

FIG. 3 is a flowchart of a physiological index detecting method.

### DETAILED DESCRIPTION

It will be appreciated that for simplicity and clarity of illustration, where appropriate, reference numerals have been repeated among the different figures to indicate corresponding or analogous elements. In addition, numerous specific details are set forth in order to provide a thorough understanding of the embodiments described herein. However, it will be understood by those of ordinary skill in the art that the embodiments described herein can be practiced without these specific details. In other instances, methods, procedures, and components have not been described in detail so as not to obscure the related relevant feature being described. The drawings are not necessarily to scale and the proportions of certain parts may be exaggerated to better illustrate details and features. The description is not to be considered as limiting the scope of the embodiments described herein.

The term “comprising” means “including, but not necessarily limited to”; it specifically indicates open-ended inclusion or membership in a so-described combination, group, series, and the like.

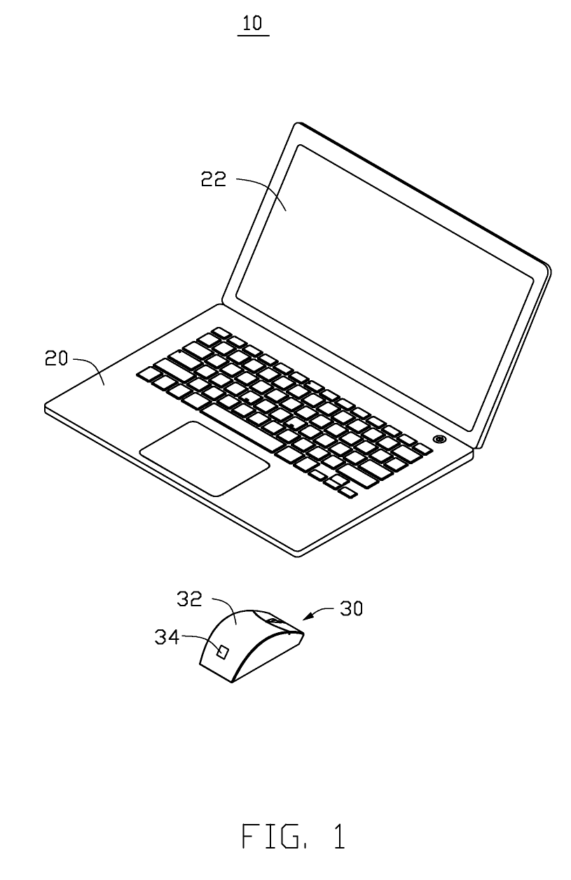

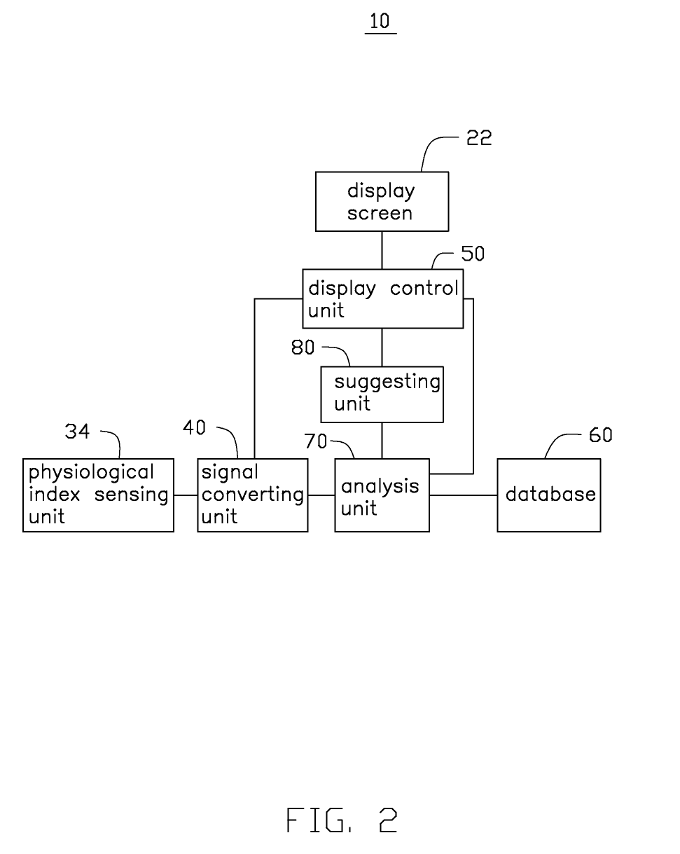

FIGS. 1 and 2 illustrate a physiological index detection system 10 employed in a computer 20. The physiological index detection system 10 includes a display screen 22 and a mouse 30 communicating with the computer 20. The physiological index detection system 10 is configured to detect a physiological index of a person who touches the mouse 30 and process the physiological index, and display a result on the display screen 22. In at least one embodiment, the computer 20 can be a notebook computer. The mouse 30 wirelessly communicates with the notebook computer.

The physiological index detection system 10 includes a physiological index sensing unit 34, a signal converting unit 40, and a display control unit 50. The physiological index sensing unit 34 is attached on a shell 32 of the mouse 30. The physiological index sensing unit 34 is configured to sense the physiological index of a living being who touches the mouse 30 and generate an analog physiological index signal, and transmit the analog physiological index signal to the signal converting unit 40. The physiological index includes heartbeat parameters and body temperature parameters. The physiological index sensing unit 34 may be a pulse sensor or/and a temperature sensor. When a person operates the computer 20 through the mouse 30 and touches the physiological index sensing unit 34, the physiological index sensing unit 34 can sense the pulse or/and the temperature of the person. The signal converting unit 40 is configured to receive the analog physiological index signal and convert the analog physiological index signal to a digital physiological index signal. The display control unit 50 is employed by the computer 20. The display control unit 50 is configured to display the physiological index of the living being on the display screen 22.

The physiological index detection system 10 further includes a database 60 and an analysis unit 70. The database 60 stores ranges of the physiological index. The analysis unit 70 is configured to analyze the physical condition of the living being according to the digital physiological index signal and the appropriate range of the physiological index. For example, the appropriate range of body temperatures is from 36° C. to 37° C. The digital physiological index signal shows a 37.5° C. body temperature. The analysis unit 70 determines that the human being is in a slightly feverish condition. The display control unit 50 is configured to display the physical condition of such a human being on the display screen 22.

The physiological index detection system 10 further includes a suggesting unit 80. The suggesting unit 80 is configured to give a medical suggestion according to the physical condition determined. For example, when the physical condition is slightly feverish, the medical suggestion is to drink more water and apply a cooling gel. The display control unit 50 is configured to display the medical suggestion on the display screen 22.

In one embodiment, the signal convert unit 40, the database 60, the analysis unit 70, and the suggesting unit 80 are all employed in the mouse 30. In another embodiment, the signal convert unit 40, the database 60, the analysis unit 70, and the suggesting unit 80 are all or partly employed in the computer 20.

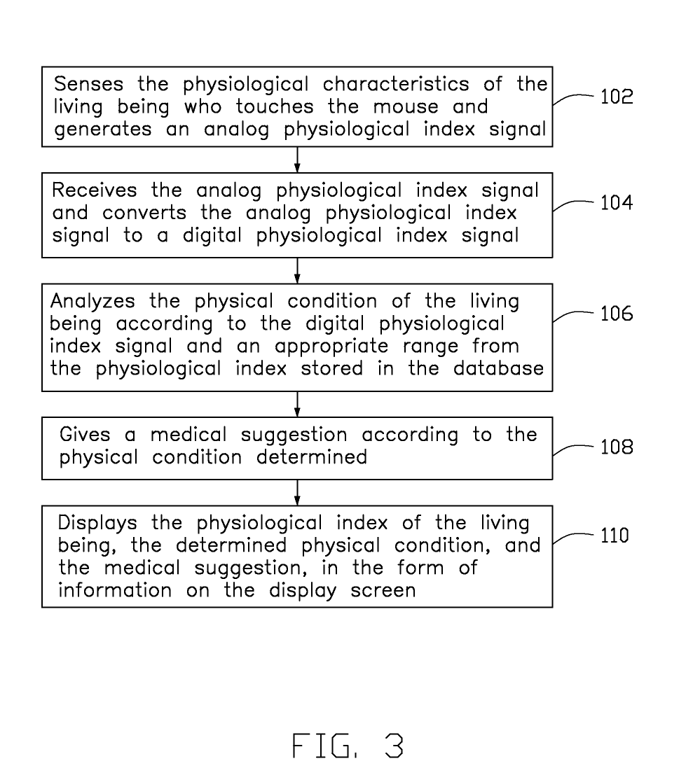

Referring to FIG. 3, a flowchart of a physiological index detecting method is presented in accordance with an example embodiment. The example method is provided by way of example, as there are a variety of ways to carry out the method. The method described below can be carried out using the configurations illustrated in FIG. 1, for example, and various elements of these figures are referenced in explaining example method. Each block shown in FIG. 2 represents one or more processes, methods, or subroutines, carried out in the example method. Furthermore, the illustrated order of blocks is illustrative only and the order of the blocks can change. Additional blocks can be added or fewer blocks may be utilized, without departing from this disclosure. The example method can begin at block 102.

At block 102, the physiological index sensing unit 34 senses the physiological characteristics of the living being who touches the mouse 30 and generates an analog physiological index signal. The physiological index sensing unit 34 is attached on a shell 32 of the mouse 30. The physiological index includes parameters regarding heartbeat and regarding body temperature.

At block 104, the signal converting unit 40 receives the analog physiological index signal and converts the analog physiological index signal to a digital physiological index signal.

At block 106, the analysis unit 70 analyzes the physical condition of the living being according to the digital physiological index signal and an appropriate range from the physiological index stored in the database 60. When the physiological index includes a body temperature the range of body temperatures within the physiological index stored in the database 60 is from 36° C. to 37° C. When the digital physiological index signal shows a 37.5° C. body temperature the analysis unit 70 determines that such a body temperature is abnormal and the person is slightly feverish.

At block 108, the suggesting unit 80 gives a medical suggestion according to the physical condition determined. When the physical condition is slightly feverish, the medical suggestion is to drink more water and apply a cooling gel.

At block 110, the display control unit 50 displays the physiological index of the living being, the determined physical condition, and the medical suggestion, in the form of information on the display screen 22.

The embodiments shown and described above are only examples. Even though numerous characteristics and advantages of the present technology have been set forth in the foregoing description, together with details of the structure and function of the present disclosure, the disclosure is illustrative only, and changes may be made in the details, including in matters of shape, size, and arrangement of the parts within the principles of the present disclosure, up to and including the full extent established by the broad general meaning of the terms used in the claims.

What is claimed is:

1. A physiological index detection system comprising:
a computer comprising a display screen and a display control unit; and
a mouse configured to communicate with the computer, the mouse comprising:
a shell;
a physiological index sensing unit attached on a shell of a mouse and configured to sense a physiological index of a living being who touches the mouse and generate an analog physiological index signal;
wherein the physiological index detection system processes the analog physiological index signal to obtain a result, and the display control unit displays the result on the display screen.

2. The physiological index detection system as claimed in claim 1, further comprises a signal converting unit, the signal converting unit receives the analog physiological index signal and converts the analog physiological index signal to a digital physiological index signal, the display control unit displays the physiological index of the living being on the display screen.

3. The physiological index detection system as claimed in claim 2, further comprises a database and an analysis unit, the database stores a range of the physiological index, the analysis unit analyzes the physical condition of the living being according to the digital physiological index signal and the appropriate range of the physiological index.

4. The physiological index detection system as claimed in claim 3, wherein the display control unit displays the physical condition of the living being on the display screen.

5. The physiological index detection system as claimed in claim 3, further comprises a suggesting unit, the suggesting unit gives a medical suggestion according to the physical condition determined.

6. The physiological index detection system as claimed in claim 5, wherein the display control unit displays the medical suggestion on the display screen.

7. The physiological index detection system as claimed in claim 6, wherein the signal convert unit, the database, the analysis unit and the suggesting unit are all employed in the computer.

8. The physiological index detection system as claimed in claim 6, wherein the signal convert unit, the database, the analysis unit and the suggesting unit are partly employed in the computer, and other part of them are employed in the mouse.

9. A mouse comprising:
a shell; and
a physiological index sensing unit attached on a shell of a mouse and configured to sense a physiological index of a living being who touches the mouse and generate an analog physiological index signal;
wherein the mouse processes the analog physiological index signal to obtain a result and transmits result to a computer.

10. The mouse as claimed in claim 9, further comprises a signal converting unit, the signal converting unit receives the analog physiological index signal and converts the analog physiological index signal to a digital physiological index signal.

11. The mouse as claimed in claim 10, further comprises a database and an analysis unit, the database stores a range of the physiological index, the analysis unit analyzes the physical condition of the living being according to the digital physiological index signal and the appropriate range of the physiological index.

12. The mouse as claimed in claim 11, further comprises a suggesting unit, the suggesting unit gives a medical suggestion according to the physical condition determined.

13. The mouse as claimed in claim 12, wherein the physiological index is a body temperature parameter.

14. The mouse as claimed in claim 13, wherein the appropriate range of the body temperature is from 36° C. to 37° C.

15. The mouse as claimed in claim 14, wherein the body temperature of the living being is 37.5° C., the analysis unit determines that the physical condition of the living being is in a slightly feverish condition.

16. The mouse as claimed in claim 15, wherein the medical suggestion is to drink more water and apply a cooling gel.

17. A physiological index detecting method comprising:
sensing a physiological characteristics of a living being who touches a mouse through a mouse and generating an analog physiological index signal;
receiving the analog physiological index signal and converting the analog physiological index signal to a digital physiological index signal;
analyzing the physical condition of the living being according to the digital physiological index signal and an appropriate range of the physiological index stored in a database; and
giving a medical suggestion according to the physical condition determined.

18. The physiological index detecting method as claimed in claim 17, further comprising:
displaying the medical suggestion on a display screen of a computer communicating with the mouse.

19. The physiological index detecting method as claimed in claim 17, further comprising:
displaying the physical condition of the living being on a display screen of a computer communicating with the mouse.

20. The physiological index detecting method as claimed in claim 17, further comprising:
displaying the physiological index of the living being on a display screen of a computer communicating with the mouse.
